(19) United States
(12) Patent Application Publication
(10) Pub. No.: US 2002/0018988 A1
(43) Pub. Date: Feb. 14, 2002

(54) In silico screening

(76) Inventors: Roscoe Klinck, Cambridge (GB); Stephen Walker, Willinghan Cambs (GB); Mohammad Afshar, Cambridge (GB); Adam Collier, Burwell Cambs (GB); Fareed Aboul-ela, Cambridge (GB); Eric Westhof, Strasbourg (FR)

Correspondence Address: PALMER & DODGE, LLP, ONE BEACON STREET, BOSTON, MA, 02108-3190, US

(21) Appl. No.: 09/843,135

(22) Filed: Apr. 26, 2001

Related U.S. Application Data
(60) Provisional application No. 60/199,773, filed on Apr. 26, 2000.

(30) Foreign Application Priority Data
Apr. 26, 2000 (GB) UK0010173.3

(51) Int. Cl. C12Q 1/70; G06F 19/00; G01N 33/48; G01N 33/50

(52) U.S. Cl. 435/5; 702/19

(57) ABSTRACT

A model structure of sub-domain IIId of the hepatitis C virus internal ribosome entry site has been elucidated. The invention provides an in silico method for identifying a compound that interacts with sub-domain IIId, comprising the steps of: (a) providing atomic co-ordinates of said sub-domain IIId in a storage medium on a Computer; and (b) using said computer to apply molecular modelling techniques to said co-ordinates. Suitable methods include de novo compound design, use of a pharmacophore, and automated docking algorithms.

This application claims the priority of U.S. Provisional Patent Application No. 60/199,773, filed on Apr. 26, 2000, and United Kingdom Patent Application UK0010173.3, filed April 26, 2000, both of which are incorporated herein in their entirety. All documents cited herein are incorporated by reference in their entirety.

### TECHNICAL FIELD

This invention is in the field of in silico screening, more particularly the use of in silico methods to identify compounds that bind to sub-domain IIId of the hepatitis C virus genome.

### BACKGROUND ART

Cap-independent translation of hepatitis C virus (HCV) genomic RNA is mediated by an internal ribosome entry site (IRES) within the 5′-UTR of the viral RNA, and inhibiting the interaction of translation initiation factors with the 5′-UTR has been proposed as a therapeutic strategy [e.g. references 1, 2 and 3].

FIG. 1 shows the secondary structure of the 5′-UTR, which is divided into four major structural domains. Domains II, III and IV contribute to IRES translational activity, and are further sub-divided into stem-loops (e.g. IIa, IIb etc.). No information concerning the tertiary structure of the IRES is presently available.

The present invention concerns sub-domain IIId (nucleotides 253-279), which has been reported as critical for IRES folding and function [4]. It is highly conserved, with only two sequence differences (co-variant alterations) between the various HCV genotypes. Sub-domain IIId is thus proposed as a drug target, and it is an object of the invention to facilitate the in silico identification and design of compounds that interact with sub-domain IIId, with a view to inhibiting IRES-mediated translation.

### SUMMARY OF THE INVENTION

The invention encompasses an in silico method for identifying a compound that interacts with sub-domain IIId of the hepatitis C virus IRES, comprising the steps of: (a) providing atomic co-ordinates of said sub-domain IIId in a storage medium on a computer; and (b) using the computer to apply molecular modelling techniques to the co-ordinates.

In one embodiment, the atomic co-ordinates are IIId_gc.pdb or IIId_gu.pdb, or variants thereof.

In another embodiment, the atomic co-ordinates are those of (i) G256, A257, G258, U259, A260, G273, A274, A275, A276 and/or (ii) U264, U265, G266, G267, G268, U269, of IIId_gc.pdb or IIId_gu.pdb.

In another embodiment, the molecular modelling techniques involve de novo compound design. In a preferred embodiment, the de novo compound design involves (i) the identification of functional groups or small molecule fragments which can interact with sites in the binding surface of sub-domain IIId, and (ii) linking these in a single compound.

In another embodiment, the molecular modelling techniques use a pharmacophore of sub-domain IIId.

In another embodiment, the molecular modelling techniques use automated docking algorithms.

In another embodiment, the compound is a reporter molecule for use in an assay for displacement from a fragment of the HCV IRES. In a preferred embodiment, the reporter molecule is a peptide, a small organic molecule, an oligonucleotide, or a PNA.

In another embodiment, the in silico method for identifying a compound that interacts with sub-domain IIId of the hepatitis C virus IRES comprises the additional steps, following step (b), of: (c) providing a compound identified by said molecular modelling techniques; and (d) contacting said compound with the HCV IRES and detecting the interaction between them.

The invention further encompasses a compound identified using the disclosed in silico method for identifying a compound that interacts with sub-domain IIId of the hepatitis C virus IRES.

The invention further encompasses a computer-readable medium for a computer, characterised in that the medium contains atomic co-ordinates of the sub-domain IIId of the hepatitis C virus IRES. In a preferred embodiment, the atomic co-ordinates are IIId_gc.pdb or IIId_gu.pdb, or variants thereof.

The invention further encompasses an assay for displacement from a fragment of the HCV IRES, wherein the assay utilises a reporter molecule identified using the methods described above.

### DETAILED DESCRIPTION OF THE INVENTION

The invention is based on the elucidation of a model structure of sub-domain IIId. This contains several unexpected structural motifs, and is readily applicable to in silico drug design.

The invention provides an in silico method for identifying a compound that interacts with sub-domain IIId of the hepatitis C virus IRES, comprising the steps of: (a) providing atomic co-ordinates of said sub-domain IIId in a storage medium on a computer; and (b) using said computer to apply molecular modelling techniques to said co-ordinates.

The atomic co-ordinates

The invention involves the use of atomic co-ordinates of sub-domain IIId. These may be co-ordinates for the complete sub-domain IIId (nucleotides 253-279), they may be co-ordinates for a fragment of the IRES that comprises sub-domain IIId, or they may be co-ordinates for a fragment of sub-domain IIId.

Preferred atomic co-ordinates for use according to the invention are IIId_gc.pdb and IIId_gu.pdb, as set out herein. Both these co-ordinate sets represent the complete 27mer sub-domain IIId. The two sets are for the two polymorphic IIId sequences found in nature, and were determined by NMR in combination with molecular modelling and phylogenetic data.

Variants of IIId_gc.pdb and IIId_gu.pdb can also be used for the invention, such as variants in which the r.m.s. deviation of the x, y and z co-ordinates for all heavy (i.e. not hydrogen) atoms are all less than 2.5 Å (e.g. less than 2 Å, preferably less than 1 Å, and more preferably less than 0.5 Å or less than 0.1 Å) compared with the structures given herein.

Preferred fragments of sub-domain IIId whose co-ordinates can be used in the invention are:

the ‘Sarcin/Ricin loop’ (SRL) motif (nucleotides A257, G258, U259, A260, G273, A274, A275);

the ‘trans-wobble’ base pair (nucleotides U264, G268); and

the terminal loop (nucleotides U264, U265, G266, G267, G268, U269).

Because of the similarity of the SRL motif to elements in human rRNA, however, a drug targeted to it may exhibit toxicity to human cells. Similarly, the terminal loop contains a fragment similar to the ‘T-loop’ of Phe-tRNA. A more preferred fragment of sub-domain IIId whose co-ordinates can be used according to the invention thus comprises both of these motifs (i.e. nucleotides A257, G258, U259, A260, U264, U265, G266, G267, G268, U269, G273, A274, A275), as their juxtaposition is not native to human RNA. The anti-anti trans-wobble U264•G268 pair in the terminal loop has not so far been observed in RNAs whose structures have been solved, offering further specificity.

The storage medium

The storage medium in which the atomic co-ordinates are provided is preferably random-access memory (RAM), but may also be read-only memory (ROM e.g. CDROM), or a diskette. The storage medium may be local to the computer, or may be remote (e.g. a networked storage medium, including the internet).

The invention also provides a computer-readable medium for a computer, characterised in that the medium contains atomic co-ordinates of sub-domain IIId of the hepatitis C virus IRES. The atomic co-ordinates are preferably IIId_gc.pdb or IIId_gu.pdb, or variants thereof.

Any suitable computer can be used in the present invention.

Molecular modelling techniques

“Molecular modelling techniques” refers to techniques that generate one or more 3D models of a ligand binding site or other structural feature of a macromolecule. Molecular modelling techniques can be performed manually, with the aid of a computer, or with a combination of these.

Molecular modelling techniques can be applied to the atomic co-ordinates of sub-domain IIId structure to derive a range of 3D models and to investigate the structure of ligand binding sites. A variety of molecular modelling methods are available to the skilled person for use according to the invention [e.g. ref. 5].

At the simplest level, visual inspection of a computer model of sub-domain IIId can be used, in association with manual docking of models of functional groups into its binding pockets.

Software for implementing molecular modelling techniques may also be used. Typical suites of software include CERIUS² [6], SYBYL [7], AMBER [8], HYPERCHEM [9], INSIGHT II [6], CATALYST [6], CHEMSITE [10], QUANTA [6]. These packages implement many different algorithms that may be used according to the invention (e.g. CHARMm molecular mechanics [11]). Their uses in the methods of the invention include, but are not limited to: (a) interactive modelling of the structure with concurrent geometry optimisation (e.g. QUANTA); (b) molecular dynamics simulation of sub-domain IIId structure (e.g. CHARMM, AMBER); (c) normal mode dynamics simulation of sub-domain IIId structure (e.g. CHARMM). As used herein “automated docking algorithm” refers to

Modelling may include one or more steps of energy minimisation with standard molecular mechanics force fields, such as those used in CHARMM and AMBER.

These molecular modelling techniques allow the construction of structural models that can be used for in silico drug design and modelling.

Some algorithmic techniques listed above are conventionally used for modelling ligand-protein interactions, but can be modified for modelling ligand-RNA interactions for use according to the present invention.

de novo compound design

De novo compound design refers to the process whereby binding surfaces of a target macromolecule (e.g., a nucleic acid or polypeptide, preferably an RNA) are determined, and those surfaces are used as a platform or basis for the rational design of compounds that will interact with those surfaces. The molecular modelling steps used in the methods of the invention may use the atomic co-ordinates of sub-domain IIId, and models derived therefrom, to determine binding surfaces. This preferably reveals van der Waals contacts, electrostatic interactions, and/or hydrogen bonding opportunities.

These binding surfaces will typically be used by grid-based techniques (e.g. GRID [12], CERIUS²) and/or multiple copy simultaneous search (MCSS) techniques [13] to map favourable interaction positions for functional groups. This preferably reveals positions in sub-domain IIId for interactions such as, but not limited to, those with protons, hydroxyl groups, amine groups, hydrophobic groups (e.g. methyl, ethyl, benzyl) and/or divalent cations. The term “functional group” refers to chemical groups that interact with one or more sites on an interaction surface of a macromolecule. A “small molecule” is a compound having molecular mass of less than 3000 Daltons, preferably less than 2000 or 1500, still more preferably less than 1000, and most preferably less than 600 Daltons. A “small molecule fragment” is a portion of a small molecule that has at least one functional group. A “small organic molecule” is a small molecule that comprises carbon.

Once functional groups or small molecule fragments which can interact with specific sites in the binding surface of sub-domain IIId have been identified, they can be linked in a single compound using either bridging fragments with the correct size and geometry or frameworks which can support the functional groups at favourable orientations, thereby providing a compound according to the invention. Whilst linking of functional groups in this way can be done manually, perhaps with the help of software such as QUANTA or SYBYL, the following software may be used for assistance: HOOK [6], which links multiple functional groups with molecular templates taken from a database, and/or CAVEAT [14], which designs linking units to constrain acyclic molecules.

Other computer-based approaches to de novo compound design that can be used with the IIId atomic co-ordinates include LUDI [15,6], SPROUT [16] and LEAPFROG [7].

Pharmacophore searching

As well as using de novo design, a pharmacophore of sub-domain IIId can be defined i.e. a collection of chemical features and 3D constraints that expresses specific characteristics responsible for biological activity. The pharmacophore preferably includes surface-accessible features, more preferably including hydrogen bond donors and acceptors, charged/ionisable groups, and/or hydrophobic patches. These may be weighted depending on their relative importance in conferring activity [17].

Pharmacophores can be determined using software such as CATALYST (including HypoGen or HipHop) [6], CERIUS², or constructed by hand from a known conformation of a lead compound. The pharmacophore can be used to screen in silico compound libraries, using a program such as CATALYST [6].

Suitable in silico libraries include the Available Chemical Directory (MDL Inc), the Derwent World Drug Index (WDI), BioByteMasterFile, the National Cancer Institute database (NCI), and the Maybridge catalog.

Docking

Compounds in these in silico libraries can also be screened for their ability to interact with sub-domain IIId by using their respective atomic co-ordinates in automated docking algorithms. An automated docking algorithm is one which permits the prediction of interactions of a number of compounds with a molecule having a given atomic structure.

Suitable docking algorithms include: DOCK [18], AUTODOCK [19,8], MOE-DOCK [20] or FLEXX [7].

Docking algorithms can also be used to verify interactions with ligands designed de novo.

Homology models

Several proteins have been identified which bind to RNAs containing elements related to the loop E motif family [reviewed in ref. 29]. They include, among others, the bacterial ribosomal protein L25 and the eukaryotic ribosomal protein L5. These proteins may bind to the SRL motif within sub-domain IIId, or can be engineered to do so, and can be used in two ways:

1. To design a reporter for a displacement assay for the identification of ligands binding to HCV sub-domain IIId. A reporter protein, or a fragment thereof, which binds to sub-domain IIId can be used in an assay for the interaction e.g. using FRET (e.g. WO99/64625), chemical footprinting, or retardation of mobility in gel electrophoresis. Compounds produced through a drug discovery program could then be assayed for their ability to disrupt this protein-RNA interaction, as an indication of binding to sub-domain IIId.

2. To design libraries of compounds for a drug discovery program targeted at binding to HCV sub-domain IIId. Whilst the native proteins and fragments may not have optimal properties for pharmaceutical use, the structure of the complex of the protein with the substrate prokaryotic loop E or SRL type RNA [cf. 21] can be used to identify elements which interact with the RNA. These elements can be mimicked by a compound (e.g. in a library designed with knowledge of structure underlying the interaction).

In both cases, the co-ordinates of the invention can be used to perfect the design as follows:

the designed reporter or compound is docked against the co-ordinates of the invention, by analogy with the interaction observed in the analogous prokaryotic loop E or SRL type motif in the known crystal or NMR structure(s);

fragments and/or functional groups from the protein which are suitable for the design of a low molecular weight compound are identified, as well as possible contacts or clashes with other parts of the IIId RNA;

the reporter or compound is then modified to alleviate steric or electrostatic clashes, reduce the molecular weight, improve pharmacological properties, and/or add favourable interactions by means described above.

Typical compounds designed in this way may be fragments from a protein, small organic molecules containing the critical functional groups, or “antisense” ligands (e.g. PNAs, oligonucleotides, etc.)

Similar methods can be used to design a reporter or compound library to interact with the terminal loop, based on analogies to the T-loop of tRNA (which interacts with the tRNA D-loop), tobramycin (which interacts with an RNA aptamer containing a U-turn [22]), or other homologous RNAs from viral or bacterial systems.

It will be appreciated that these techniques can be applied to any RNA which contains these structural motifs, not just sub-domain IIId of the HCV IRES.

‘Dual site’ design

A compound identified using the invention preferably interacts with one or more nucleotides from the ‘loop E’ motif (A257, G258, U259, A260, G273, A274, A275) and one or more nucleotides from the terminal loop (U264, U265, G266, G267, G268, U269). These two regions contain homologies to human RNA structures and, as it is believed that sub-domain IIId functions in vivo by mimicking these structures and thereby sequestering cellular proteins, a compound that interacts with only one of these two regions may be toxic to the host. As the juxtaposition of these motifs appears to be unique to HCV, however, targeting them both simultaneously will allow specificity. Moreover, the U264•G268 pair adds further specificity.

In general, the design strategy begins by searching for ligands with relatively weak affinity to each of these two sites. Linking these two ligands in order to permit their simultaneous interaction with the target typically increases affinity by orders of magnitude. Moreover, the RNA regions between the terminal loop and the loop E motif contain distinctive features which can be recognised by an appropriate linker, such as the U264•G268 pair, adding further specificity and affinity.

Basis for further models

The atomic co-ordinates of the invention can be used as the basis of models of further RNA structures. For example, a homology model of a RNA structure could be based on the sub-domain IIId structures of the present invention.

Furthermore, the structures of fragments of the sub-domain IIId model can be used as the basis of modelling equivalent structures in other RNA molecules. Where a RNA molecule is thought to contain a loop E motif, for instance, the structure of nucleotides A257, G258, U259, A260, G273, A274, & A275 of HCV sub-domain IIId can be used as a template. Similarly, the ‘trans-wobble’ base pair (nucleotides U264, G268) of sub-domain IIId can be used as the basis of a model.

Testing compounds

The methods of the invention may comprise the further steps of: (c) providing a compound identified by said molecular modelling techniques; and (d) contacting said compound with the HCV IRES and assaying the interaction between them.

Suitable methods for assaying the interaction between the HCV IRES and the compound include: (i) the direct methods disclosed in WO99/64625; (b) the indirect methods disclosed in references 23 and 24. Preferred indirect methods use bicistronic constructs containing two different luciferases, the first being translated in a cap-dependent manner and the second being translated from the HCV IRES in a cap-independent manner. The relative levels of the two luciferases gives an indication of whether the IRES-mediated translation was inhibited.

Compounds and their uses

The methods of the invention identify compounds that can interact with sub-domain IIId of the hepatitis C virus IRES. These compounds may be designed de novo, may be known compounds, or may be based on known compounds.

The invention also provides: (i) a compound identified using the methods of the invention; (ii) a compound identified using the methods of the invention for use as a pharmaceutical; (iii) the use of a compound identified using the methods of the invention in the manufacture of a medicament for treating hepatitis C infection; and (iv) a method of treating a patient with hepatitis C infection, comprising administering an effective amount of a compound identified using the methods of the invention.

### BRIEF DESCRIPTION OF DRAWINGS

FIG. 1 shows the HCV TRES, including secondary structural motifs. Sub-domain IIId is enlarged and boxed.

FIG. 2 shows the construct used to assess mutant IRES activity.

FIG. 3 shows various motifs from the ‘loop E’ or ‘SRL’ family. 3A shows the sarcin/ricin (SRL) loop and chemical shifts, and 3B shows the prokaryotic 5S rRNA loop E. In 3C, these structures are mapped onto HCV sub-domain IIId.

FIG. 4 shows example NMR spectra from sub-domain IIId. 4A shows the region of a NOESY (150 ms mixing time) spectrum acquired in H₂O, illustrating NOEs between imino proton and aromatic/amino proton resonances. 4B shows the region of a NOESY (120 ms mixing time) spectrum acquired in D₂O, illustrating NOEs between aromatic proton resonances, with the characteristic NOE between the H2 proton resonances of A260 and A2274 highlighted. 4C shows a natural-abundance ¹H-¹³C HMQC spectrum. Positive peaks (predominantly to the left) are darker than the negative peaks (predominantly to the right).

FIG. 5 highlights NMR signals from the terminal loop region. 5A shows the imino-imino proton region of NOESY in H₂O. 5B shows the aromatic to anomeric proton NOESY at 400 ms which establish the relative geometries of nucleotides G268 and C270.

FIG. 6 shows the two sub-domain IIId sequences for which 3D structures were produced.

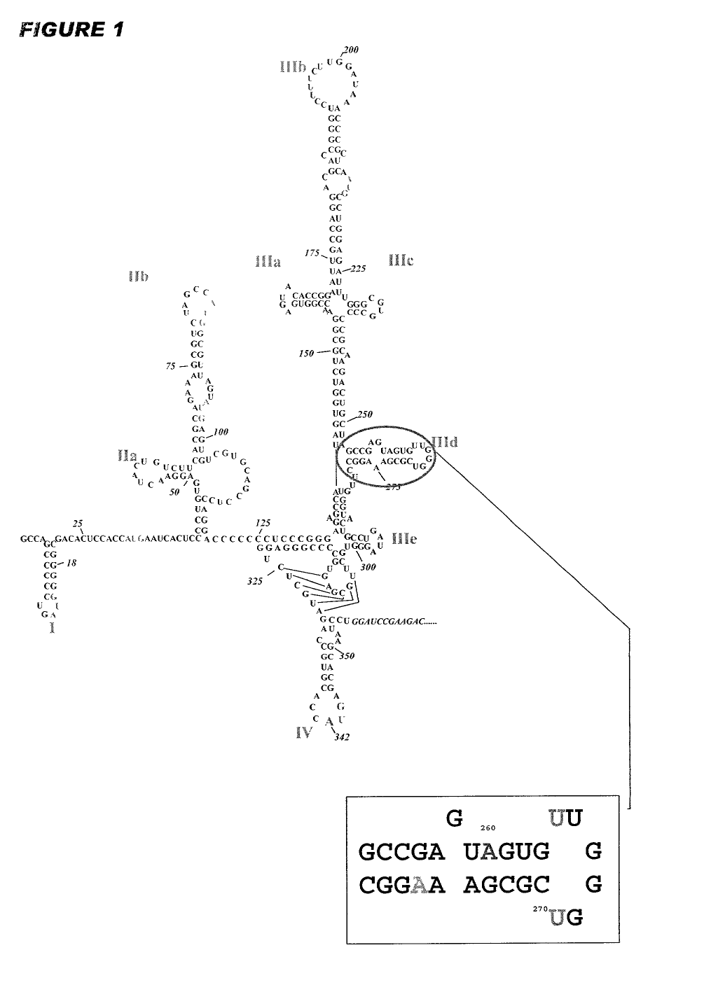

### MODES FOR CARRYING OUT THE INVENTION

Mutational analysis of sub-domain IIId

Substitution mutants of sub-domain IIId were created using oligonucleotide site-directed mutagenesis using the Stratagene QuickChange Kit™. The template plasmid was pTZ18:5442-16-1, which contains the HCV Ia 5′-UTR (nucleotides 18-357) in the BamHI site of pTZ18U [25]. Mutant derivatives of the 5′-UTR were sub-cloned into the BamHI site of dual reporter pRT9, constructed as follows: (i) pRT1 was constructed by deleting the BamHI site of pRL-Nu11 (Promega) using Klenow; (ii) the EcoRV/HindIII fragment of pD5(3.3) [26] was inserted into Bg1II(blunt)/HindIII digested pRT1, to give pRT2; (iii) the SacI/HindIII blunt-ended fragment of pRL-5442-16-1 [24] was inserted into pRT2 digested with NheI/XbaI and blunt-ended.

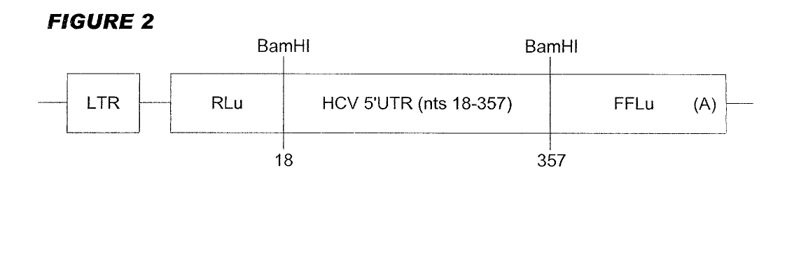

pRT9 thus contains the HIV-1 LTR (nucleotides −340 to +78) and transcribes a bicistronic mRNA encoding renilla luciferase, the HCV 5′-UTR, and firefly luciferase (FIG. 2). The ratio of renilla and firefly luciferases indicates the activity of the IRES in the 5′-UTR.

To assay the relative activities, 35 cm² dishes were seeded with 2×10⁵ C63 HeLa cells. After 24 hours, transfections were performed with 1 μg pRT9 (or mutant) using CaPO₄. After 40 hours, cells were harvested, lysed and assayed for both luciferases using the Promega dual-luciferase assay (following manufacturer's instructions), and results were as follows:

| Mutant | | IRES activity (%) |
|---|---|---|
| Wild-type control | | 100 |
| Loop (264-269) | ²⁶⁴UUGGGU CUUCGG | 7 |
| AA (275-276) | ²⁷⁵AA CUC | 25 |
| ²⁶⁰A | C | 40 |
| | G | 12 |
| | U | 47 |
| | CG | 1 |
| ²⁷⁶A | U | 57 |
| | G | 45 |
| | GU | 7 |
| ²⁶⁴U:²⁶⁹U | G:G | 25 |
| | C:A | 27 |
| | A:C | 42 |
| | A:A | 27 |
| | C:C | 27 |
| | G:A | 18 |
| | G:C | 27 |
| | C:G | 18 |

Replacing the terminal hexaloop (264-269) with a tetraloop sequence abolishes IRES activity. To investigate this in further detail, dual substitution mutants for ²⁶⁴U and ²⁶⁹U were constructed and, consistent with the tetraloop mutant, IRES activity was significantly diminished.

Replacing the AA dinucleotide (275-276) with CUC, thereby converting the internal loop into a double helix, also reduced IRES activity. Single point mutants at residues 260 and 276 within the internal loop, gave similar results, as did insertions.

The terminal and internal loops in IIId are thus crucially important for IRES activity.

Modelling the IIId structure—hypothesis

The internal loop within IIId contains a sequence almost identical to that in the sarcin/ricin loop fragment [27], which forms a ‘loop E’ motif structure [e.g. 28, 29, 30, 31, 32, 33, 34]. It was thus hypothesised that the internal loop of IIId would fold in the same manner.

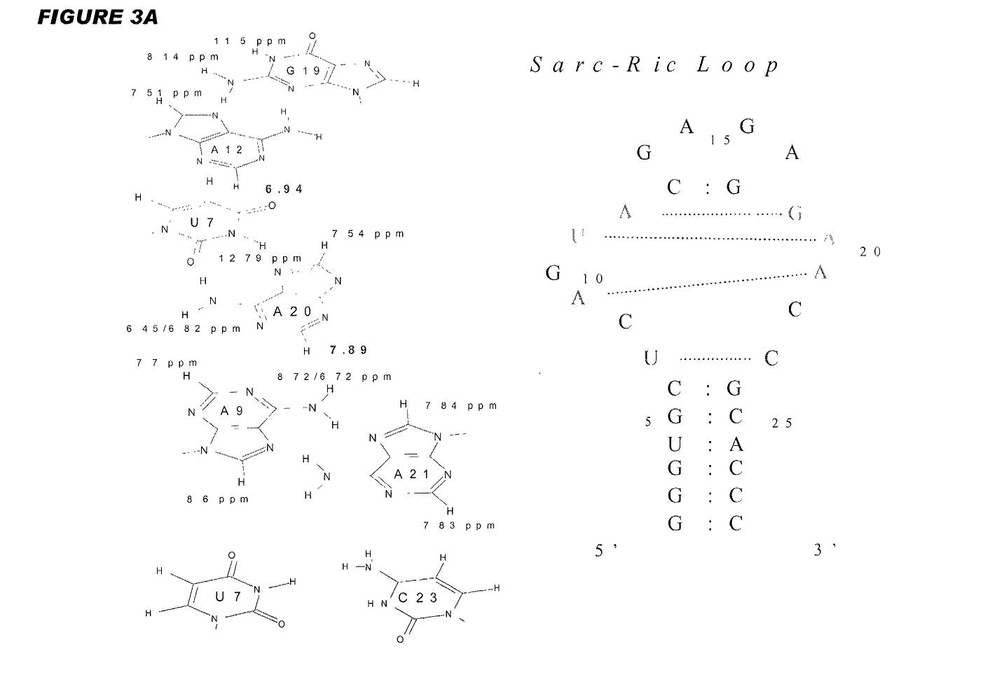

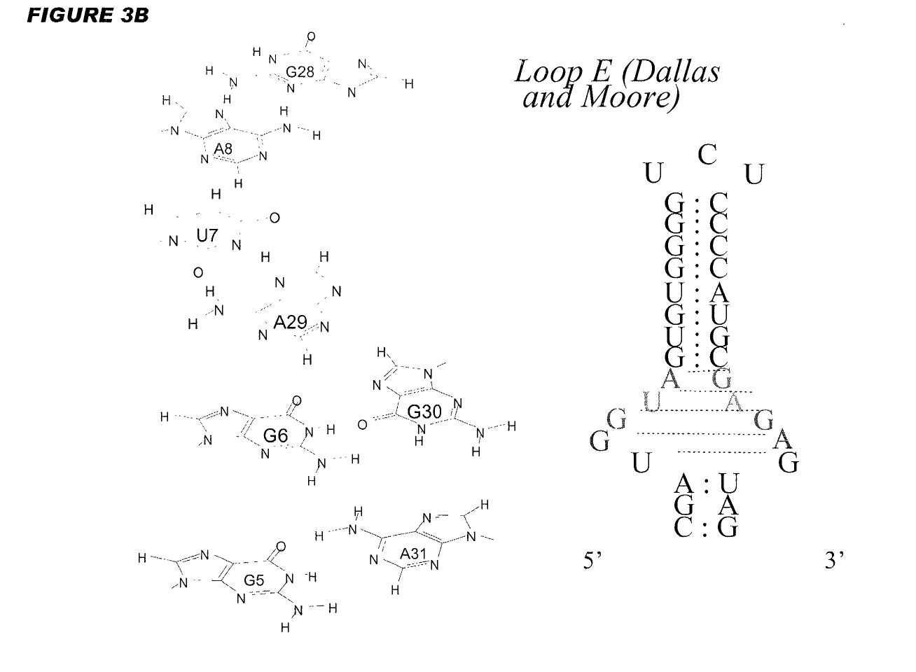

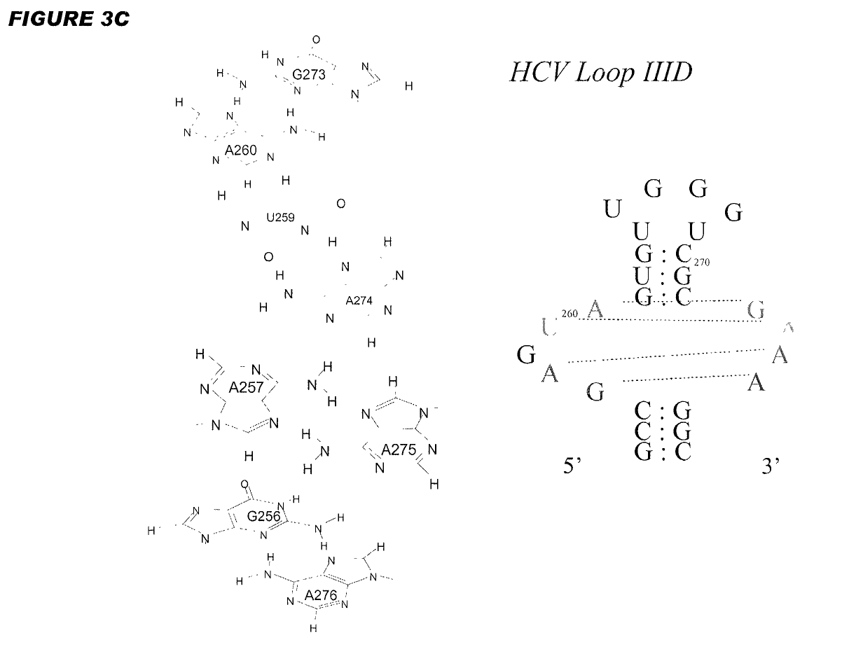

Examples of the ‘loop E’ or ‘SRL’ motif family have been observed in the eukaryotic 5S rRNA loop E and in the sarcin/ricin loop (FIG. 3A) from rRNA, where it is universally conserved. Another example is present in the prokaryotic 5S rRNA loop E (FIG. 3B), but this lacks the bulged-out nucleotide and, furthermore, is present as tandem copies.

The SRL motif within the sarcin/ricin loop itself gives rise to a number of unusual chemical shifts (FIG. 3A): 8.6 ppm for the H8 proton of A9, 81.3 ppm for the C1′ resonance of G10, and just over 4 ppm for the anomeric protons of the C13•G18 base pair. In addition, distinct pairs of amino proton resonances appear associated with punines in the SRL motif. Amino resonances from A9 appear at 8.72 ppm and 6.72 ppm, similar to shifts observed for amino resonances in Watson-Crick paired regions, but the A20 aminos resonate at 6.45 ppm and 6.82 ppm. Distinctive NOE patterns are predicted from the non-canonical base-pairing schemes shown in FIG. 3A e.g. the reverse trans-Hoogsteen U11•A20 base pair will give rise to NOEs between the imino proton of U11 and the H8 and amino protons of A20. Based on the hypothesis, analogous NOEs were predicted between U259 and A274 of sub-domain IIId. In addition, an exceptionally intense cross-strand NOE is seen in the SRL between the H2 protons of A20 and A12, due to the unusual geometry of the motif. An analogous NOE between A260 and A274 of sub-domain IIId was predicted. The reversal in strand direction between A10 and U12 in the SRL is known as an ‘S-motif’ or ‘S-turn’.

NMR was used to test the hypothesis.

NMR spectra

A 27mer RNA identical in sequence to sub-domain IIId of the HCV IRES was synthesised by T7 RNA polymerase transcription from synthetic DNA templates [35]. Transcripts were purified on 20% polyacrylamide gels containing 7M urea [36], and full-length transcripts were excised from the gels, electro-eluted, and dialysed into 8 mM sodium phosphate buffer, pH 6.6. Addition of sodium or magnesium chloride has no significant effect on the NMR spectra, and was thus not included in the sample buffer. Final concentration in NMR samples was 1.2 mM RNA in 200 μl volume.

NMR spectra were recorded on Bruker DRX500 and DMX600 spectrometers. For analysis of exchangeable protons, NOESY experiments were run at 5° C. and 25° C. A jump-return-WATERGATE sequence was used for water suppression [37]. The sample was lyophilised and re-suspended in D₂O for non-exchangeable proton assignment. NOESY (60, 120, 150 and 400 ms mixing times), TOCSY and COSY-DQF experiments were run at 20° C. and 30° C. Proton-phosphonts and proton-carbon (natural abundance) carbon heteronuclear correlation experiments, ¹H-³¹P-COSY, ¹H-³¹P-hetero-TOCSY and ¹H-¹³C-HMQC were performed at 30° C. Proton chemical shifts were referenced to the residual water peak (4.77 ppm at 25° C.).

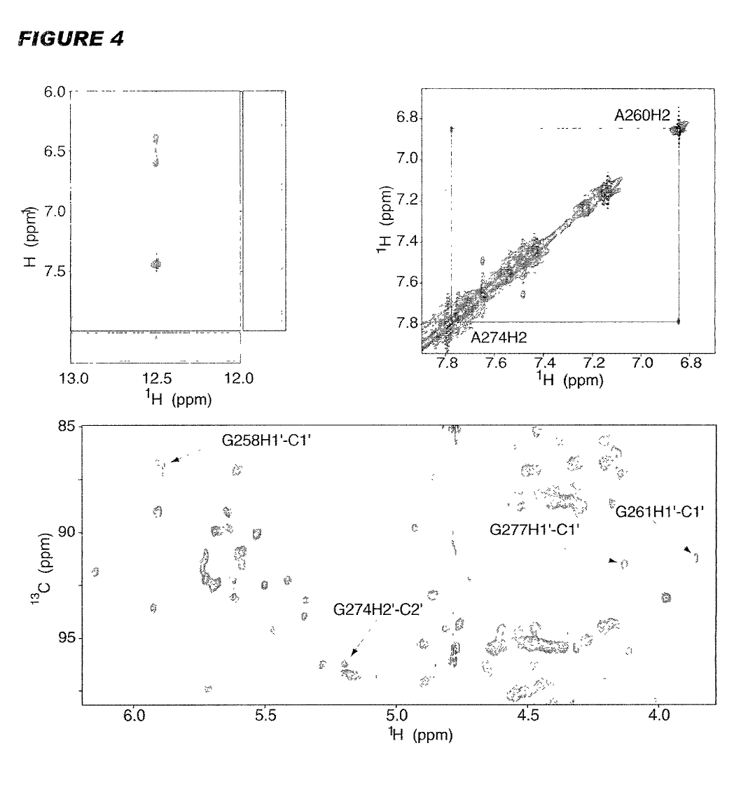

As shown in FIG. 4A, a pair of NOE signals appears near 6.5 ppm, arising from amino resonances of A274. A sharp cross-peak near 7.5 ppm demonstrates a close contact between U259 imino (12.5 ppm) and A274 H8 (7.5 ppm), confirming the predicted trans-Hoogsteen base pair. At the contour level shown in FIG. 4B, two aromatic-aromatic NOEs are visible. The A274 H2 to A260 H2 NOE cross-peak, analogous to the intense cross-strand NOE (A20 & A12) seen in the SRL, is indicated. In FIG. 4C, two anomalous H1′ proton shifts (G261 & G277 H1′) and one unusual C1′ carbon shift (G258 C1′) are seen in positions strikingly similar to those observed in the SRL. Together, these data confirm that the overall fold and the base pairings in this region of IIId are the same as those of the SRL. The hypothesis was correct.

Additional data from NMR spectra

As well as confirming the SRL motif for the IIId internal loop, the NMR spectra suggested further structural elements.

U269 bulge

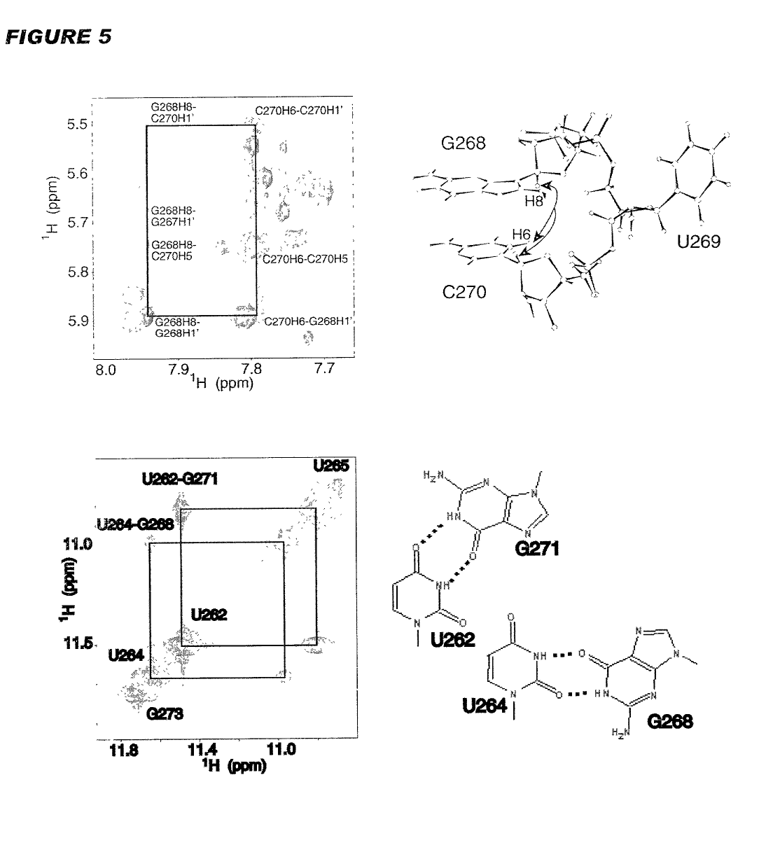

The aromatic to anomeric protein NOE connectivity path in IIId is broken between nucleotides G268 and C270, which show NOEs to each other (i.e. base stacking) in both directions (FIG. 5A). A similar ‘box pattern’ of NOEs can arise where alternating anti and syn glycosidic angles are present, as in Z-DNA [38] or ‘foldback’ G-quartet structures [39]. In the absence of the diagnostic signals associated with syn glycosidic geometry, however, this pattern can only arise in IIId from a localised backbone inversion at G268. This inversion in accommodated by, and dependent upon, the bulged-out nucleotide U269, indicated by U269 only presenting intranucleotide cross-peaks.

The presence of a locally inverted nucleotide 5′ to a bulged-out nucleotide has been reported in several structures, including the RRE [40] and the loop E motif of the SRL.

G268•U264 ‘trans-wobble’ base pair

The formation of a G268•U264 base pair is indicated by imino proton resonances at 10.9 ppm (G268H1) and 11.6 ppm (U264H3), as shown in FIG. 5B. Considering the backbone inversion at the position of G268, this base pair must be of a locally parallel trans type, involving hydrogen bonding between G268H1 & U264O4 and G268O6 & U264H3. Both of these imino resonances show NOEs to each other and to G263H1, consistent with stacking of the U264•G268 pair on the G263•C270 pair. A ‘trans-wobble’ G•U base pair has been observed previously in the crystal structure of a fragment of the hepatitis delta virus ribozyme, albeit with the G in the syn conformation [41].

‘U-turn’ motif

With U264 and G268 forming a base pair, the backbone turn in the IIId terminal loop must be accomplished by U265, G266 and G267 only. These three residues are predicted to form a ‘U-turn’ motif [42,43].

Sharp turns in nucleic acid helices require major distortions in backbone torsion angles from those found in helical regions. A set of characteristic torsion angles was observed in the first tRNA crystal structures, especially within loop regions. The distortion is localised at the α and ξ torsion angles in three phosphate residues in the loop. Similar results have been seen in crystal and NMR structures of RNA [44, 45, 46, 47]. This motif is referred to as the ‘U-turn’, and is often associated with a uracil residue which stacks on the i+2 phosphate (the ‘stacking phosphate’) while hydrogen bonding to oxygen on the i+3 phosphate (the ‘H-bonding phosphate’).

All observed proton-proton NOEs for IIId are consistent with this motif, and the U265-P-G266, G266-P-G267 and G267-P-G268 chemical shifts are in the predicted order relative to each other.

Conclusion

Overall, there are no unassigned imino, amino or aromatic resonances, the presence of which would indicate the formation of alternative or unfolded structures. In isolation, the 27mer IIId fragment forms an exceptionally stable secondary structure, which is likely to be maintained within the context of the full HCV 5′-UTR. In particular, ‘loop E’ motifs seen in rRNA are maintained in the presence of ribosomal proteins [48], and ‘U-turn’ motifs seen in tRNAs and in the GTPase centre hexaloop are maintained in the presence of tertiary interactions with other RNA loops.

Modelling sub-domain IIId

The NMR data was used in conjunction with a motif-based approach in order to construct a model of the three-dimensional structure of sub-domain IIId. Six motifs were used:

| | |
|---|---|
| (i) | an A-form double helix |
| (ii) | a sheared G.A base pair |
| (iii) | a SRL motif |
| (iv) | a localised backbone inversion |
| (v) | a trans-wobble G.U base pair |
| (vi) | a U-turn |

The presence of each of these six motifs has experimental basis in the NMR spectra.

Examples of motifs (ii)-(iv) & (vi) were extracted from NMR and crystal PDB structures. Motifs (ii) and (iii) were taken from the SRL structure [430D.pdb, ref. 49]. Motif (iv) was extracted from the RRE structure [1ETG.pdb, ref 40]. Motif (vi) was extracted from the GTPase RNA structure [1QA6.pdb, ref. 47].

Motif (i) was built using idealised co-ordinates (InsightII biopolymer module [6]), and motif (v) was generated with InsightII using idealised base planarity and hydrogen-bonding distances and angles.

The motifs were ligated together in silico as follows. The G253 to C255 double helix was constructed using InsightII. A sheared G256•A276 base pair was added manually using InsightII, maintaining acceptable C25503′-G256P and G277P-A276O3′ distances. The A257-A260 and G273-A275 loop E motif was then positioned. Idealised A-form co-ordinates were then used to build the G261-G263 double helix, with some manual adjustment to incorporate the U262•G271 base pair. The backbone inversion at G268/U269 was then positioned in such a way as to optimise G268-C270 stacking and to allow a suitable G268 orientation for the positioning of U264. Using A-form co-ordinates, U264 was positioned to stack on G263 and form a trans-wobble pair with G268. The U-turn motif was then positioned to complete the loop sequence between U264 and G268. All the components of the model were ligated using the InsightII biopolymer module and the resulting structure was energy minimised using Charmm 25.a2 to remove unfavourable bond lengths and angles.

The resulting structure is given below as IIId_gc.pdb.

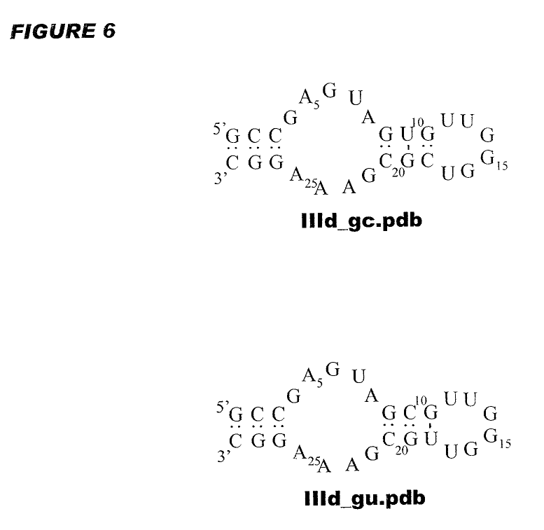

Taking into account the natural polymorphism in HCV, the same procedure was followed for a sub-domain IIId having the sequence ²⁶²CGUUGGGUUG²⁷¹ instead of ²⁶²UGUUGGGUCG²⁷¹ (see FIG. 6). This structure is given below as IIId_gu.pdb.

These models were carefully analysed to ensure conformity with the NMR spectra. The A-form helix and loop E motif could be directly compared with published NMR data and were in extremely good agreement. A list of all probable and improbable NOEs expected from these terminal loop region of the model was compared to the NMR data and in all cases the model and NMR were consistent. In addition, the U269 orientation in the models is consistent with it presenting only intranucleotide NOEs.

The 3D models were constructed in a fraction of the time that would have been required for a de novo NMR or crystal structure determination, but the end product is of excellent quality and is suitable for use in molecular modelling and in silico drug design.

It will be understood that the invention has been described by way of example only and modifications may be made whilst remaining within the scope and spirit of the invention.

1. An in silico method for identifying a compound that interacts with sub-domain IIId of the hepatitis C virus IRES, comprising the steps of: (a) providing atomic co-ordinates of said sub-domain IIId in a storage medium on a computer; and (b) using said computer to apply molecular modelling techniques to said co-ordinates.

2. The method of claim 1, wherein the atomic co-ordinates are IIId_gc.pdb or IIId_gu.pdb, or variants thereof.

3. The method of claim 1, wherein the atomic co-ordinates are those of (i) G256, A257, G258, U259, A260, G273, A274, A275, A276 and/or (ii) U264, U265, G266, G267, G268, U269, of IIId_gc.pdb or IIId_gu.pdb.

4. The method of claim 1, wherein the molecular modelling techniques involve de novo compound design.

5. The method of claim 4, wherein the de novo compound design involves (i) the identification of functional groups or small molecule fragments which can interact with sites in the binding surface of sub-domain IIId, and (ii) linking these in a single compound

6. The method of any one of claims 1 to 3, wherein the molecular modelling techniques use a pharmacophore of sub-domain IIId.

7. The method of any one of claims 1 to 3, wherein the molecular modelling techniques use automated docking algorithms.

8. The method of claim 1, wherein the compound is a reporter molecule for use in an assay for displacement from a fragment of the HCV IRES.

9. The method of claim 8, wherein the reporter molecule is a peptide, a small organic molecule, an oligonucleotide, or a PNA.

10. The method of claim 1, comprising the additional steps, following step (b), of: (c) providing a compound identified by said molecular modelling techniques; and (d) contacting said compound with the HCV IRES and detecting the interaction between them.

11. A compound identified using the method of claim 1.

12. A computer-readable medium for a computer, characterised in that the medium contains atomic co-ordinates of the sub-domain IIId of the hepatitis C virus IRES.

13. The medium of claim 12, wherein the atomic co-ordinates are IIId_gc.pdb or IIIdg_gu.pdb, or variants thereof.

14. An assay for displacement from a fragment of the HCV IRES, wherein the assay utilises a reporter molecule identified using the method of claim 8 or claim 9.
